(19) United States
(12) Patent Application Publication
(10) Pub. No.: US 2006/0069366 A1
(43) Pub. Date: Mar. 30, 2006

(54) Core for an absorbent article

(76) Inventor: Robert Theodore Cole, Jackson, NJ (US)

Correspondence Address: RATNERPRESTIA, P O BOX 980, VALLEY FORGE, PA, 19482-0980, US

(21) Appl. No.: 10/950,101

(22) Filed: Sep. 24, 2004

(51) Int. Cl. A61F 13/15

(52) U.S. Cl. 604/378

(57) ABSTRACT

A core is provided for an absorbent article comprising an absorbent layer having first and second sides, the absorbent layer having a central portion and a perimeter portion. The core also comprises an acquisition layer disposed adjacent to the absorbent layer, with the acquisition layer having a central portion and a perimeter portion. The central portion of the acquisition layer is disposed adjacent the first side of the central portion of the absorbent layer and the perimeter portion of the acquisition layer is disposed adjacent the second side of the perimeter portion of the absorbent layer.

### FIELD OF THE INVENTION

This invention relates to a core for an absorbent article and a method of making a core for an absorbent article.

### BACKGROUND OF THE INVENTION

Absorbent articles such as diapers, training pants, panty liners, adult incontinence devices, and other absorbent products are well known in the art. Typically, these articles comprise liquid handling members which are specifically designed for the acquisition of fluids disposed onto the article in use.

There have been ongoing efforts to improve the performance of such absorbent articles. For example, it has been recognized in the prior art that it is beneficial for the wearing comfort of such an article to transport the acquired fluid away from the point of acquisition. Providing sufficient void space in the liquid handling member below the point of acquisition requires the liquid acquisition member to have a high caliper and hence to be very bulky. This problem can only be avoided if the acquired fluid is transported away from the point of acquisition in a direction parallel to the surface of the liquid handling member.

As a result, it may be observed in some absorbent articles that only a small fraction of the x, y-dimension of the liquid handling member is actually used when a surge of fluid is acquired. Hence, the absorbent and liquid acquisition performance of the liquid handling member is often not fully utilized.

Despite efforts to improve absorbent articles, there continues to be a need for absorbent articles that can be configured for improved performance.

### SUMMARY OF THE INVENTION

The invention includes a core for an absorbent article comprising an absorbent layer having first and second sides, the absorbent layer having a central portion and a perimeter portion. The core also comprises an acquisition layer disposed adjacent to the absorbent layer, with the acquisition layer having a central portion and a perimeter portion. The central portion of the acquisition layer is disposed adjacent the first side of the central portion of the absorbent layer and the perimeter portion of the acquisition layer is disposed adjacent the second side of the perimeter portion of the absorbent layer.

Another embodiment of the invention includes a method of making an absorbent article having an acquisition layer and an absorbent layer. The method comprises the steps of: (a) separating central and perimeter portions of the absorbent layer; and (b) interposing the acquisition layer between the central and perimeter portions of the absorbent layer such that a central portion of the acquisition layer is adjacent a side of the central portion of the absorbent layer and a perimeter portion of the acquisition layer is adjacent an opposite side of the absorbent layer in the perimeter portion of the absorbent layer.

Yet another embodiment of the invention includes a method of making a core for an absorbent article comprising the steps of: (a) forming a central portion of an absorbent layer; (b) positioning an acquisition layer adjacent to the central portion of the absorbent layer, such that a perimeter portion of the acquisition layer extends from the central portion of the absorbent layer; and (c) positioning a perimeter portion of the absorbent layer adjacent the perimeter portion of acquisition layer.

In another embodiment of the invention, regarding an absorbent article having an acquisition layer and an absorbent layer, a method of making the absorbent article includes the steps of: (a) separating central and perimeter portions of the absorbent layer; (b) interposing the acquisition layer between the central and perimeter portions of the absorbent layer such that a central portion of the acquisition layer is adjacent a side of the central portion of the absorbent layer and a perimeter portion of the acquisition layer is adjacent an opposite side of the absorbent layer in the perimeter portion of the absorbent layer; (c) trimming outer edges of the absorbent and acquisition layers to form a core; and (d) interposing the core between a top-sheet and a back-sheet.

### BRIEF DESCRIPTION OF THE DRAWINGS

Exemplary embodiments of this invention will be described with reference to the drawing, of which:

FIG. 1 is a top view of an exemplary embodiment of a core configured for use in an absorbent article according to an exemplary embodiment of the present invention.

FIG. 2 is a bottom view of the core of FIG. 1.

FIG. 3 is a cross-sectional view of the core of FIG. 1.

FIG. 4 is a cross-sectional view of an absorbent article according to an exemplary embodiment of the present invention.

FIG. 5 is a cross-sectional view of an absorbent article according to another exemplary embodiment of the present invention.

FIG. 6 is a top view schematic illustrating an embodiment of a process for forming a core for an absorbent article according to an exemplary embodiment of the present invention.

FIG. 7 is a cross-sectional schematic illustrating the process of FIG. 6.

FIG. 8 is a flow chart of exemplary process steps for forming a core for an absorbent article according to an exemplary embodiment of the present invention.

### DETAILED DESCRIPTION OF THE INVENTION

The invention is best understood from the following detailed description when read in connection with the accompanying drawing, which shows exemplary embodiments of the invention selected for illustrative purposes. It is emphasized that, according to common practice, the various features of the drawing are not to scale. On the contrary, the dimensions of the various features may be arbitrarily expanded or reduced for clarity.

According to one exemplary embodiment, the invention includes a core of an absorbent article. The core comprises an absorbent layer having a central portion and a perimeter portion. The core also contains an acquisition layer disposed adjacent to the absorbent layer.

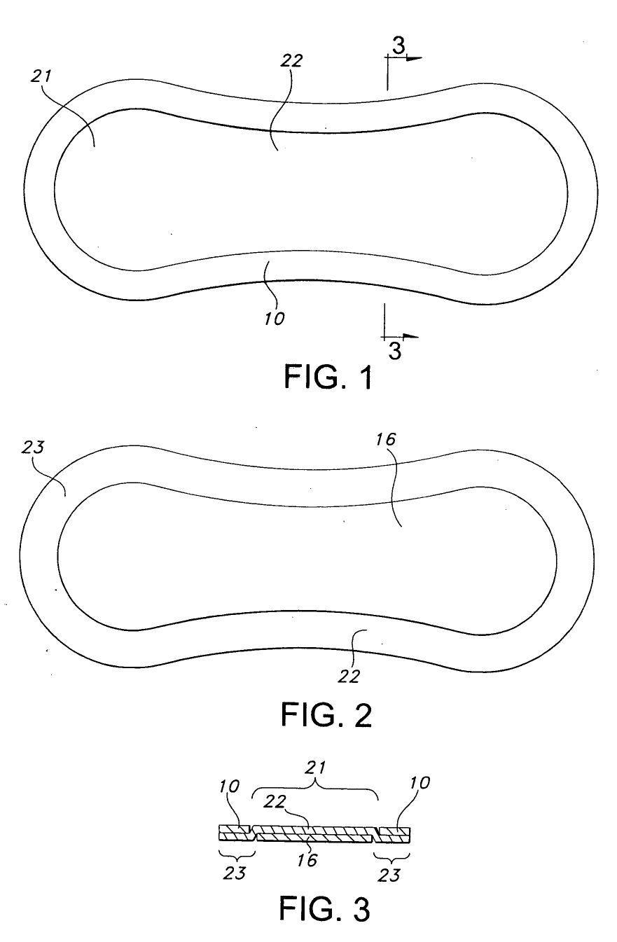

Referring generally to the drawing (FIGS. 1-8), a core is provided for use in an absorbent article. The core includes an absorbent layer having first 20 and 12 and second 18 and 14 sides, the absorbent layer having a central portion 16 and a perimeter portion 10. The central portion of the absorbent layer having a first side 20 and a second side 18, and the perimeter portion of the absorbent layer having a first side 12 and a second side 14. The core also includes an acquisition layer 22 disposed adjacent the absorbent layer 10, 16, the acquisition layer 22 having a central portion 21 and a perimeter portion 23. The central portion 21 of the acquisition layer is disposed adjacent the first side 20 of the central portion of the absorbent layer, and the perimeter portion 23 of the acquisition layer is disposed adjacent the second side 14 of the perimeter portion of the absorbent layer.

The absorbent article is formed by separating central 16 and perimeter 10 portions of the absorbent layer. The acquisition layer 22 is then interposed between the central and perimeter portions of the absorbent layer such that a central portion 21 of the acquisition layer is adjacent a side of the central portion 16 of the absorbent layer and a perimeter portion 23 of the acquisition layer is adjacent an opposite side of the absorbent layer in the perimeter portion 10 of the absorbent layer.

Referring now to the drawing in particular, in which like reference numbers refer to like elements throughout, FIG. 1 shows a top view of a core of an absorbent article. In the top view, the perimeter portion 10 of the absorbent layer is shown, and the acquisition layer 22 is shown in the central region of the core. FIG. 2 shows a bottom view of the same core shown in FIG. 1. In this view, the central portion 16 of the absorbent layer is viewable, as well as the acquisition layer 22 in the perimeter region of the core.

The shape of the absorbent core is optionally dog bone shaped as illustrated in FIGS. 1 and 2. Alternatively, any shape can be selected depending upon the type and configuration of the absorbent article in which the core is intended to be used. Also, although the shapes of the central and perimeter portions of the core are optionally the same as illustrated in FIGS. 1 and 2, the shapes of the central and perimeter portions of the core can be different depending again on the type and configuration of the absorbent article in which the core is intended to be used.

While exemplary embodiments of the invention discussed herein refer to an absorbent layer positioned adjacent an acquisition layers, this invention also contemplates structures having other combinations of layers. For example, this invention contemplates an absorbent structure having multiple absorbent layers, multiple acquisition layers, or combinations of multiple absorbent and acquisition layers.

To illustrate, it is contemplated that an absorbent structure may have two absorbent layers positioned adjacent one another and optionally having different absorbencies (e.g., two airlaids of different densities or basis weight). In such an embodiment, a lower density absorbent layer is optionally positioned over an absorbent layer of higher density, making it possible for the lower density layer to disburse fluid and the higher density layer to wick the fluid in a perimeter region.

Also, an absorbent structure according to this invention optionally has three or more layers. Such layers may be provided by a combination of one or more absorbent layers, one or more acquisition layers, or other types of layers typically found in absorbent articles.

FIG. 3 shows a cross sectional view of the core shown in FIG. 1. As shown in FIG. 3, the perimeter portion 10 of the absorbent layer is positioned above the acquisition layer 22, while the central portion 16 of the absorbent layer is positioned below the acquisition layer 22. The invention is not limited to this configuration, as the relative positioning of the components may be inverted, with the perimeter portion 10 of the absorbent layer positioned below the acquisition layer 22, while the central portion 16 of the absorbent layer is positioned above the acquisition layer 22.

As shown in FIG. 3, the acquisition layer 22 is continuous and interposed between the perimeter portion 10 of the absorbent layer and the central portion 16 of the absorbent layer. Although not illustrated in the drawings, the invention also includes variations of the shown configuration, including a segmented acquisition layer interposed between multiple regions of the absorbent layer.

In contrast to the acquisition layer shown in FIG. 3, the absorbent layer is discontinuous between its central 16 and perimeter 10 portions. The absorbent layer may contain additional segments, having two or more portions of absorbent layer interposed by the acquisition layer. In additional embodiments of the invention, the absorbent layer may be continuous and interposed between segments of the acquisition layer.

The portions of the absorbent layer can be made from any suitable absorbent material, as well as combinations of different types of absorbent materials. For example, in one embodiment of the invention, the absorbent layer is formed of an air-laid absorbent material, such as wood pulp, which can optionally contain a super absorbent polymer powder (SAP) and a binder. Examples of SAP include polyacrylamides, polyvinyl alcohol, polyacrylates, various grafted starches, and the like. One particularly suitable super absorbent material is a cross-linked polysodium acrylate, which can be purchased from BASF Corporation, Portsmouth, Va., under the trade designation 2100A. For clarity, the absorbent layer is shown in the figures as a single layer, however, the absorbent layer may comprise multiple components functioning as an absorbent system.

A fluid acquisition layer serves to manage, transport, accommodate and/or direct high volumes and flow rates of fluid into the core. The fluid acquisition layer can be of any type construction, e.g., a thru-air bonded/carded web, a spunbond bicomponent non-woven web, a web of crosslink cellulosic fibers, apertured 3D (three dimensional) film, adhesive bonded fibers, or the like. For clarity, the acquisition layer is shown in the figures as a single layer, however, the acquisition layer may comprise multiple components functioning as an acquisition system.

The core shown in FIG. 3 optionally has a substantially constant thickness. Alternatively, the core may be thicker or thinner in various regions depending on its intended use.

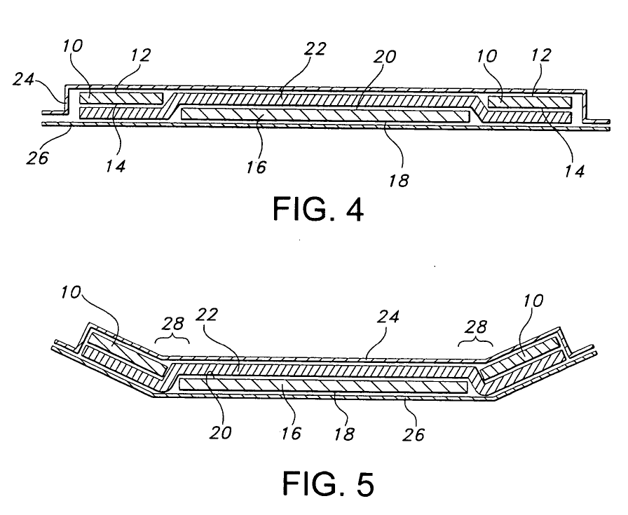

In another embodiment, the core is incorporated into an absorbent article, such as a diaper, training pant, incontinence pad, feminine hygiene product, or other absorbent product. An exemplary absorbent article is illustrated in a cross-sectional view in FIG. 4. Similar to the core shown in FIG. 3, the acquisition layer 22 is interposed between the perimeter portion 10 of the absorbent layer and the central portion 16 of the absorbent layer. Additionally, a top-sheet 24 is positioned above the core, adjacent the first side 12 of the perimeter portion 10 of the absorbent layer and adjacent the acquisition layer 22 in the central region of the core. The core, as shown in FIG. 4, includes the acquisition layer 22 adjacent the second side 14 of the perimeter portion 10 of the absorbent layer. The acquisition layer 22 is adjacent the first side 20 of the central portion 16 of the absorbent layer. As shown in FIG. 4, the top-sheet 24 is continuous between the central portion of the acquisition layer and the perimeter portion of the absorbent layer.

The top-sheet 24 may be of any liquid permeable material such as material fibers (e.g., polyethylene, bi-component, polyester, rayon, cotton, etc.), fiber combinations (e.g., spunbond, air laid, wet laid, carded, thermal bonded, hydroentangled, etc.), and basis weights as well. One particularly suitable material is a 13.5 gsm wettable non-woven coverstock, made of spun bond polypropylene, available from AVGOL Nonwoven Industries LTD., Holon, Israel. If desired, the top-sheet 24 may be formed of a liquid impermeable material, e.g., three dimensional polymeric film, having plural apertures or pores extending therethrough so as to make the material liquid permeable. The top-sheet 24 is disposed on top of the core and can be secured thereon by a hot melt adhesive.

The absorbent article may also include a back-sheet. As shown in FIG. 4, a back-sheet 26 is adjacent the perimeter portion of the acquisition layer 22, and adjacent the second side 18 of the central portion 16 of the absorbent layer. The back-sheet 26 is continuous between the central portion 16 of the absorbent layer and the perimeter portion of the acquisition layer, and is generally made of a liquid impervious material. The back-sheet 26 may be the same shape as the cover sheet 24, and adhesively bonded to the cover sheet to encapsulate the core.

The back-sheet 26 is may be formed of a laminated sheet of a non-woven material and film (with the non-woven side positioned as the outermost layer). Such material should be hydrophobic, soft in texture, and strong in tensile strength. One particularly suitable material is a spunbond-meltblown-spunbond (SMS) web having a basis weight of about 15 gms per square meter (gsm), available from AVGOL Nonwoven Industries LTD., Holon, Israel. The spunbond layer is made of polypropylene fibers. Such composites provide the dual advantages of liquid barrier properties of film along with a soft, arm outer fabric texture. The non-woven outer back-sheet can also be made of other suitable cloth-like materials, e.g., spun-bond or thermal-bond non-woven web made of either polypropylene, polyethylene, polyester, bi-component fibers (polyethylene/polypropylene or polyethylenelpolyester), or any combinations of these fibers. Various multiple layer configurations or fiber denier variations may be used. Another example includes hydro-entangled non-woven webs, which may contain some cotton and/or rayon fibers blending in with thermal-plastic fibers. Cellulose fibers can also be blended in at small percentages to reduce cost.

Still another example is a non-woven outer cover made of stretchable or elastic materials, such as elastomeric composites of non-woven(s) and elastic membranes or a single layer of elastic material. The elastomeric composite can comprise of an inner layer of pre-stretched extruded elastic film sandwiched between and attached to a pair of non-woven webs. The non-woven webs may consist of spun-bond web, thermal-bond web, or a combination of the two. Preferably, the elastic film is made of synthetic rubber and the non-woven made of spun-bond polypropylene.

Other materials for forming the back-sheet 26 may include polypropylene films, co-extruded films (polyethylene and ethylene vinyl acetate), co-polymer films (polyethylene/polypropylene), and polylaminates (polypropylene nonwoven and polyethylene film). Still another example is a film made of a “breathable” microporous polyethylene. Suitable breathable films are available from Exxon Chemical Company, Buffalo Grove, Ill. This material allows water vapor to pass through it over time, while being impervious to liquid water. The water vapor transmission rate may range from 200-4000 grams per square meter per 24-hour period.

Although the absorbent article embodiment illustrated in FIG. 4 includes an absorbent layer, an acquisition layer, a top-sheet, and a back-sheet, an absorbent article according to this invention may include fewer or more components. For example, an absorbent article is optionally provided with fastening mechanisms, elastic components, gathers, tabs, and other components depending on the intended use of the article. Also, one or both of the top-sheet and bottom-sheet are optionally eliminated.

FIG. 5 presents a cross-sectional view of another embodiment of an absorbent article that has a natural concave shape. An absorbent article containing a core according to the present invention is optionally configured to develop a concave shape upon manufacture or upon fluid absorption. The interposition of the absorbent and acquisition layers allow the perimeter portions to curl inward in response to the manufacturing process or in response to fluid absorption in use. This effect improves performance of the absorbent article in reducing the occurrence of leaks from the edges of the absorbent article. The degree of curvature of the article can be modified by adjusting the relative dimensions of the perimeter and central portions of the layers of the core.

The invention promotes the absorption of fluids that may be transferred from the central areas (typically the location of fluid insult during use) to the perimeter region of the core. Such transport of fluids laterally to the perimeter of the core is generally undesirable when it could exceed the absorbent capacity of the core and result in leakage at the perimeter. For this reason, and because the acquisition layer of an absorbent article can promote the transfer of fluid radially outwardly from the area of insult, the acquisition layers of conventional absorbent articles generally do not extend to the outer perimeter of the core or to the outer perimeter of the absorbent layer.

According to exemplary embodiments of this invention, however, the acquisition layer optionally extends to a location near the perimeter edge of the core (or all the way to the perimeter edge as illustrated in exemplary embodiments). It has been discovered that this structure can actually improve absorption by the absorbent layer, as the perimeter portion of the absorbent layer serves to provide additional absorbency. In exemplary embodiments of the present invention, for example, the perimeter portion of the absorbent layer is available to absorb fluids that may be transferred toward the perimeter of the core by the acquisition layer. The interposition of the absorbent and acquisition layers therefore encourages the absorption of fluids lateral transferred to the perimeter portion of the absorbent layer or core during use of the absorbent article. Such absorption at the perimeter tends to prevent leakage of fluids from the article and further utilizes the absorbent capacity of the article.

Efficient access to the perimeter portion of the absorbent layer is achieved by transporting fluids via the acquisition layer through a separation in the absorbent layer. The transposition of the acquisition layer through the portions of the absorbent layer permits fluid transfer through both sides of the acquisition layer, as both the outward facing side, and the inward facing side of the acquisition layer are in contact with the absorbent layer. By facilitating the absorption of fluids by the perimeter portion of the absorbent layer, the core avoids user discomfort and leakage resulting from fluid absorption concentrated in the center of the core.

It has been discovered that acquisition layers (and multi-voids of such acquisition layers) can, in some circumstances, allow excess fluid to migrate across the acquisition layer in all directions, thereby attacking the lateral sides of a product before the absorbent core is fully utilized. Typically, therefore, a traditional acquisition layer is not placed or sized in such a way as to extend to the edge of an absorbent product.

An interposed joint or interposition region 28 (see FIG. 5) is therefore provided according to one exemplary aspect of the invention. Such interposition region 28 facilitates a desired deformation of the product and controls fluid migration. In anatomical terms, the acquisition layer acts as a ligament between portions of the absorbent layer in that it holds the joint together yet allows it to bend. During use, the lateral edge of the absorbent product will tend to bend upward (as illustrated in FIG. 5), thereby forcing the fluid to be absorbed into the perimeter absorbent barrier.

The present invention also includes methods of manufacturing cores and absorbent articles. Specifically included is a method of making an absorbent article comprising the steps of: (a) separating central and perimeter portions of the absorbent layer; and (b) interposing the acquisition layer between the central and perimeter portions of the absorbent layer such that a central portion of the acquisition layer is adjacent a side of the central portion of the absorbent layer and a perimeter portion of the acquisition layer is adjacent an opposite side of the absorbent layer in the perimeter portion of the absorbent layer.

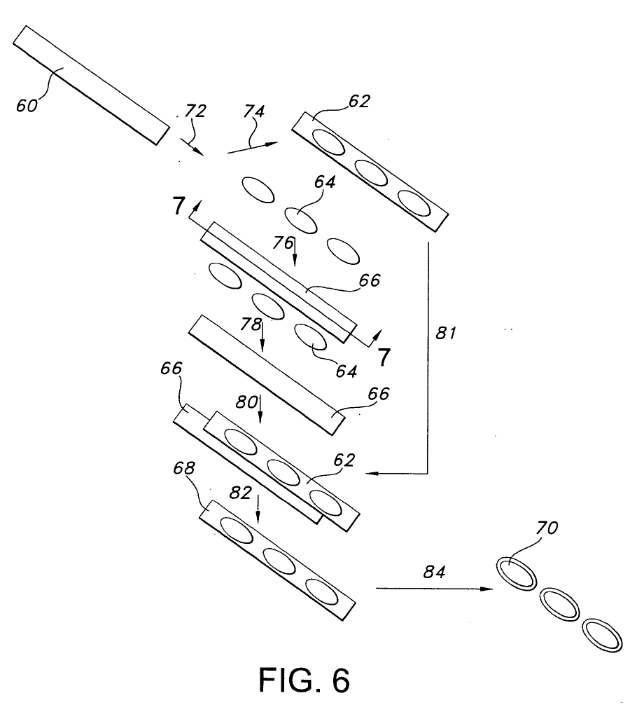

FIG. 6 illustrates the process of assembling the core of an absorbent article from sheets of component materials. For illustration purposes, FIG. 6 presents the assembly of three cores simultaneously in a single row. The methods of the present invention are adaptable to configurations encompassing the assembly of single cores as well as multiple cores simultaneously in single or multiple rows or other configurations, and such configurations are limited only by the machinery and materials employed in the assembly process.

As shown in FIG. 6, the central portion of the absorbent layer 64 is die cut from a sheet of absorbent material 60. The sheet of absorbent material remaining 62 after the central portion of the absorbent core is cut out is temporarily spaced from the central portion or removed from the assembly process, while the central portion of the absorbent layer 64 is covered by a sheet of acquisition layer material 66. With the acquisition layer material positioned adjacent the central portion of the absorbent layer cut-out 64, the remaining sheet of absorbent material 62 is positioned over the sheet of acquisition layer material 66.

The three components 62, 66, 64 are combined to form a reciprocal core sheet 68, and the core 70 is die-cut from the reciprocal core sheet. Preferably, the core die-cut dimensions extend beyond the dimension of central portion of the absorbent layer dimensions. By extending beyond the central portion of the absorbent layer, absorbent material from the remaining sheet 62 of absorbent material placed above the acquisition layer material 66 forms the perimeter portion of the absorbent layer.

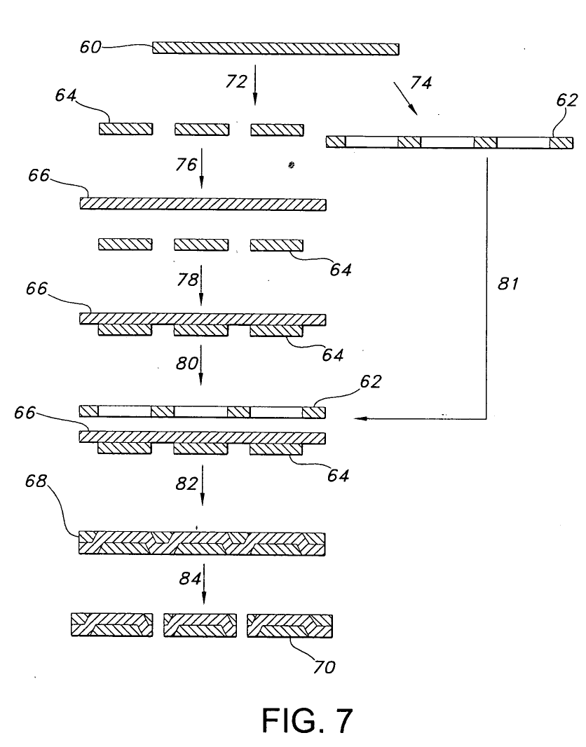

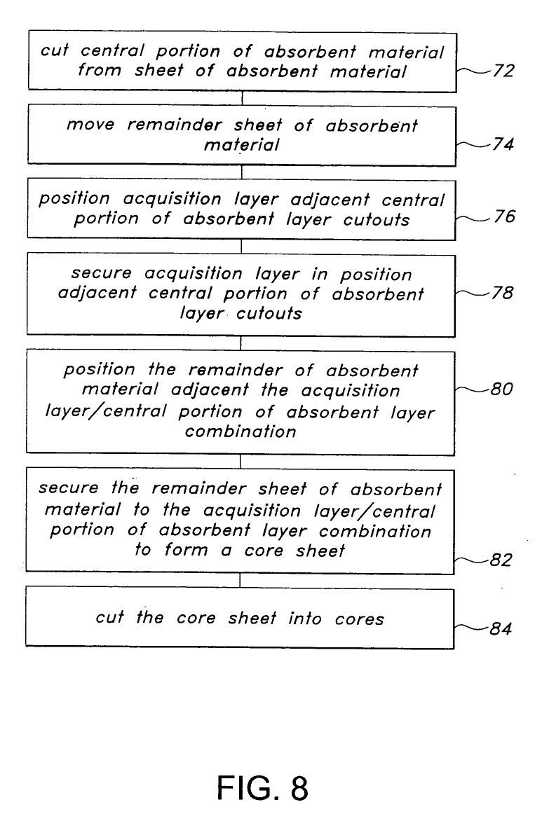

FIG. 7 provides a cross-sectional view of the process illustrated in FIG. 6, and a flowchart of the process steps is provided in FIG. 8. The process steps include die-cutting the central portion of absorbent material from a sheet of absorbent material 72. The cut-out 64 provides the central portion 16 of the absorbent layer of the core. The sheet of absorbent material remaining after the die-cut 62 is temporarily removed or separated from the assembly 74, and the acquisition layer material 66 is positioned adjacent the central portion of the absorbent layer cut-outs 76. The acquisition layer material is secured in position 78, and the remaining sheet of the absorbent material 62 is positioned adjacent the acquisition layer material sheet 80. The three components are secured in position 82 to form a reciprocal core sheet 68, and the reciprocal core sheet is die-cut 84 to form the core.

While not shown in the figures, additional steps for the process of making an absorbent article include positioning a top-sheet over the core, optionally adjacent the perimeter portion of the absorbent layer and adjacent the acquisition layer on the side opposite the side of the acquisition layer adjacent to the central portion of the absorbent layer, i.e. the top side of the core as it is illustrated in the figures. Additionally, in the process of making an absorbent article, a back-sheet can be positioned on the bottom of the core, as depicted in the figures. For example, the back-sheet can be positioned adjacent the central portion of the absorbent layer and adjacent the acquisition layer in the perimeter portion of the core. The top-sheet and back-sheet can be adhered to each other, to the core, or to both in an outer perimeter area.

The figures include the following reference numbers, provided with a summarizing label for convenience.

10 absorbent layer perimeter portion

12 first side of absorbent layer perimeter portion

14 second side of absorbent layer perimeter portion

16 absorbent layer central portion

18 second side of absorbent layer central portion

20 first side of absorbent layer central portion

21 central portion of acquisition layer

22 acquisition layer

23 perimeter portion of acquisition layer

24 top-sheet

26 back-sheet

28 interposition region

60 sheet of absorbent material

62 remainder of sheet of absorbent material after cutting step

64 central portion of absorbent layer

66 sheet of acquisition material

68 core sheet

70 core

72 step of cutting central portion of absorbent material

74 step of removing remainder sheet of absorbent material

76 step of positioning acquisition layer adjacent central portion of absorbent layer cutouts

78 step of securing acquisition layer in position over central portion of absorbent layer cutouts

80 step of positioning the remainder sheet of absorbent material over the acquisition layer/central portion of absorbent layer combination

82 step of securing the remainder sheet of absorbent material to the acquisition layer/central portion of absorbent layer combination to form a core sheet

84 step of cutting the core sheet into cores

Although the invention is illustrated and described herein with reference to specific embodiments, the invention is not intended to be limited to the details shown. Rather, various modifications may be made in the details within the scope and range of equivalents of the claims and without departing from the invention.

What is claimed:

1. A core for an absorbent article comprising:
an absorbent layer having first and second sides, said absorbent layer having a central portion and a perimeter portion; and
an acquisition layer disposed adjacent said absorbent layer, said acquisition layer having a central portion and a perimeter portion;
said central portion of said acquisition layer being disposed adjacent said first side of said central portion of said absorbent layer and said perimeter portion of said acquisition layer being disposed adjacent said second side of said perimeter portion of said absorbent layer.

2. The core of claim 1 wherein the acquisition layer is continuous between the central portion of the acquisition layer and the perimeter portion of the acquisition layer.

3. The core of claim 1 wherein the absorbent layer is discontinuous between the central portion of the absorbent layer and the perimeter portion of the absorbent layer.

4. The core of claim 1 wherein the acquisition layer extends between the perimeter portion of the absorbent layer and central portion of the absorbent layer.

5. The core of claim 1 wherein the absorbent layer comprises a super-absorbent polymer (SAP).

6. An absorbent article comprising the core of claim 1, the absorbent article further comprising a top-sheet positioned adjacent the first side of the perimeter portion of the absorbent layer.

7. The absorbent article of claim 6 wherein the top-sheet is positioned adjacent the central portion of the acquisition layer.

8. The absorbent article of claim 6 wherein the top-sheet is formed of a liquid permeable material.

9. An absorbent article comprising the core of claim 1, the absorbent article further comprising a back-sheet positioned adjacent the second side of the central portion of the absorbent layer.

10. The absorbent article of claim 9 wherein the back-sheet is positioned adjacent the perimeter portion of the acquisition layer.

11. The absorbent article of claim 9 wherein the back-sheet is formed of a liquid impervious material.

12. In an absorbent article having an acquisition layer and an absorbent layer, a method of making an absorbent article comprising the steps of:
(a) separating central and perimeter portions of the absorbent layer; and
(b) interposing the acquisition layer between the central and perimeter portions of the absorbent layer such that a central portion of the acquisition layer is adjacent a side of the central portion of the absorbent layer and a perimeter portion of the acquisition layer is adjacent an opposite side of the absorbent layer in the perimeter portion of the absorbent layer.

13. A method of making a core for an absorbent article, the method comprising the steps of:
(a) forming a central portion of an absorbent layer;
(b) positioning an acquisition layer adjacent to the central portion of the absorbent layer, such that a perimeter portion of the acquisition layer extends from the central portion of the absorbent layer; and
(c) positioning a perimeter portion of the absorbent layer adjacent the perimeter portion of the acquisition layer.

14. The method of claim 13, said positioning step (c) comprising positioning the perimeter portion of the absorbent layer adjacent a side of the acquisition layer opposite a side of the acquisition layer adjacent the central portion of the absorbent layer.

15. The method of claim 13, said forming step (a) comprising cutting the central portion of the absorbent layer from a sheet of absorbent material.

16. The method of claim 15, said positioning step (c) comprising positioning the sheet of absorbent material adjacent the deposited acquisition layer.

17. The method of claim 13 further comprising the step of:
(d) positioning a top-sheet adjacent the perimeter portion of the absorbent layer and adjacent the acquisition layer on a side opposite a side of the acquisition layer adjacent to the central portion of the absorbent layer.

18. The method of claim 17 further comprising the steps of:
(e) positioning a back-sheet adjacent the central portion of the absorbent layer and adjacent the acquisition layer on a side opposite a side of the acquisition layer adjacent to the perimeter portion of the absorbent layer; and
(f) adhering a perimeter of the top-sheet to the back-sheet.

19. The method of claim 13 further comprising the step of:
(d) positioning a back-sheet adjacent the central portion of the absorbent layer and adjacent the acquisition layer on a side opposite a side of the acquisition layer adjacent to the perimeter portion of the absorbent layer.

20. In an absorbent article having an acquisition layer and an absorbent layer, a method of making an absorbent article comprising the steps of:
(a) separating central and perimeter portions of the absorbent layer;
(b) interposing the acquisition layer between the central and perimeter portions of the absorbent layer such that a central portion of the acquisition layer is adjacent a side of the central portion of the absorbent layer and a perimeter portion of the acquisition layer is adjacent an opposite side of the absorbent layer in the perimeter portion of the absorbent layer;
(c) trimming outer edges of the absorbent and acquisition layers to form a core; and
(d) interposing the core between a top-sheet and a back-sheet.
